(19) United States
(12) Patent Application Publication
(10) Pub. No.: US 2006/0222613 A1
(43) Pub. Date: Oct. 5, 2006

(54) Hair treating agent

(75) Inventor: Kentaro Teramoto, Osaka-shi (JP)

Correspondence Address: ARMSTRONG, KRATZ, QUINTOS, HANSON & BROOKS, LLP, 1725 K STREET, NW, SUITE 1000, WASHINGTON, DC, 20006, US

(73) Assignee: MILBON CO., LTD., Osaka-shi (JP)

(21) Appl. No.: 11/095,535

(22) Filed: Apr. 1, 2005

(51) Int. Cl. A61K 8/64

(52) U.S. Cl. 424/70.5

(57) ABSTRACT

The present invention provides a hair treating agent that is applied after applying a reducing agent for straightening curly hair in straightening treatment of curly hair and uniformly softens hair without damaging. Specifically, the present invention relates to a hair treating agent that is applied after applying a reducing agent for straightening curly hair to end reducing reaction and enables visual observation of the ending of the reaction, comprising a pH buffer and an oxyacid metal salt.

### BACKGROUND OF THE INVENTION

The present invention relates a hair treating agent that is applied after applying a reducing agent for straightening curly hair.

There are many kinds of curly hair of different degrees of curling, from wavy hair to frizzy hair, and of different degrees of damage due to recent coloring and blow drying. The process of straightening such curly hair is referred to as straightening treatment of curly hair. The reaction with hair in straightening treatment of curly hair is exactly the same as in permanent wave treatment. Commercialization of the preparation used for straightening curly hair has been allowed by the Japanese Pharmaceutical Affairs Law in 1985 and since then, various methods for straightening curly hair have been employed until the method of straightening curly hair using a high temperature hair-styling iron was reached.

The method commonly used in straightening treatment of curly hair is the method of straightening using a two bath-type straightening agent for curly hair. In this method, curly hair is straightened by the following process. The hair is thoroughly combed and arranged so as not to become tangled and then, after the reducing agent for straightening curly hair is applied to each block, the reducing agent is left (hair is softened) according to the type of hair. Then, after the hair is confirmed to have lost its elasticity and become soft (softened state), combing using a rough comb is conducted so as to stretch the hair. If the reaction is insufficient and straightening of the curly hair is insufficient, the hair is reapplied with the reducing agent for straightening curly hair and combed. Subsequently, the reducing agent for straightening curly hair is rinsed off and the hair is dried. Then, an oxidizing agent for straightening curly hair is applied to each block in the same manner as in straightening treatment of curly hair. The hair is combed with a rough comb while applying tension and the oxidizing agent is rinsed off.

The basic composition of the reducing agent for straightening curly hair is largely divided into the active ingredient (reducing agent), the auxiliary agent (alkali agent), the additive and water. The reducing agent which is the active ingredient of the reducing agent for straightening curly hair is necessary for reducing and disconnecting the cystine bonds of hair and as the reducing agent, thioglycolic acid, ammonium thioglycolate and L-cysteine are often used. By rendering the reducing agent alkaline by the alkali agent, which is the auxiliary agent, the reducing power of the reducing agent is increased. The swelling degree of hair is also increased on the alkaline side and therefore, the solution penetrates easily. As a result, the action of the reducing agent to the hair also increases, thereby increasing the straightening force. On the other hand, on the acidic side, the reducing force is extremely small and the hair cannot be sufficiently straightened (see JP-2002-363041 and JP-A-5-117134).

The aspect of the above process to which the most attention must be paid is the time for hair softening treatment. For example, when hair that is not sufficiently softened is stretched with a comb, the desired effect cannot be obtained. On the other hand, in the case of curly hair of a strong curling degree, a reducing agent for straightening curly hair having high pH containing a large amount of an alkali agent must be used to sufficiently soften the hair and therefore, damage to the hair, such as breaking at the roots and dry feel, is extremely large. The time for softening treatment differs depending on the type of hair that is subjected to the treatment and the reducing agent composition for straightening curly hair that is used, but when softening is conducted for an excessive reaction time (over time), the hair is modified, leading to damage. For example, hair that has been repeatedly colored is largely susceptible to damage at the ends and because the protein inside the hair has escaped, the hair is more susceptible to external physical influence. When such hair is subjected to softening treatment, there are areas that are softened and areas that are not. Usually, in such a case, the reducing agent for straightening curly hair must be reapplied, left and at times, heated. However, while conducting these steps, the areas that are already softened are reacted excessively and damaged significantly. That is, uniformly softening hair without damaging has been believed to be extremely difficult.

In order to solve the above problems, complicated processes were required, such as varying the time of leaving the reducing agent for straightening curly hair and applying reducing agents for straightening curly hair of different reducing power according to the damaged areas and healthy areas. Thus, the operational efficiency is extremely poor and also, there is the problem that experience is necessary to conduct the process.

### SUMMARY OF THE INVENTION

The present invention aims to provide a hair treating agent that is applied after applying a reducing agent for straightening curly hair in straightening treatment of curly hair.

As a result of intensive studies to solve the above problems, a hair treating agent containing a pH buffer and an oxyacid metal salt was found to end the reducing reaction of the reducing agent for straightening curly hair to areas where the reducing action is to be suppressed and to enable visual observation of the ending of the reaction. Thus, the present invention was reached.

That is, the present invention relates to a hair treating agent that is applied after applying a reducing agent for straightening curly hair to end reducing reaction and enables visual observation of the ending of the reaction, comprising a pH buffer and an oxyacid metal salt.

The pH of the hair treating agent is preferably 2 to 4.

The pKa of the acid composing the pH buffer is preferably 2 to 4.

The oxyacid metal salt is preferably copper gluconate and the amount thereof is 0.1 to 10% by weight.

### DETAILED DESCRIPTION

The present invention relates to a hair treating agent that is applied after applying a reducing agent for straightening curly hair to end reducing reaction and enables visual observation of the ending of the reaction, comprising a pH buffer and an oxyacid metal salt.

The pH buffer used in the present invention is composed of acid and salts thereof. The acid is not particularly limited, but is preferably an acid having dissociation constant pKa at 25° C. of 2 to 4. When pKa is less than 2, the buffer action tends to be insufficient and when pKa is more than 4, the touch of the hair after treatment tends to be rough. Examples of such acids are malic acid (pKa=3.46), glycollic acid (pKa=3.83), lactic acid (pKa=3.86), citric acid (pKa=3.04), phosphoric acid (pKa=2.15) and formic acid (pKa=3.75). Examples of salts thereof are alkali metal salts such as sodium salt and potassium salt, alkali earth metal salts and ammonium salt. The acid and salts thereof that compose the pH buffer can be a mixture of a plurality of the acids and salts thereof. The amount of the buffer in the hair treating agent is preferably 1 to 30% by weight, more preferably 5 to 20% by weight. When the amount is less than 1% by weight, the buffer action tends to be insufficient and when the amount is more than 30% by weight, the touch of the hair after treatment tends to be rough and also, the hair tends to lack gloss.

From the viewpoint of ending the reducing action of the reducing agent for straightening curly hair at the acidic side, the hair treating agent of the present invention preferably has pH of 2 to 4, more preferably 2 to 3. When the pH is more than 4, the effect of suppressing the reducing action of the reducing agent in the straightening process tends to be small and when the pH is less than 2, the skin and hair tend to be damaged.

The hair treating agent of the present invention must be acidic and ending of the reducing reaction cannot be visually observed with indicators such as phenolphthalein, which has the color changing region on the alkali side, and methyl orange, which has the color changing region of the acidic side. However, this problem does not occur in the case of an oxyacid metal salt, which is not dependent on pH for the color changing method. The oxyacid metal salt of the present invention preferably comprises an oxyacid selected from the group consisting of gluconic acid, lactic acid, ascorbic acid, citric acid, malic acid and glycollic acid and a metal selected from the group consisting of copper, zinc, iron and calcium. From the viewpoint that the change in color is more noticeable, copper gulconate is particularly preferable. The amount of the oxyacid metal salt is preferably 0.1 to 10% by weight, more preferably 0.5 to 5% by weight. When the amount is less than 0.1% by weight, the change in color tends to be unclear and when the amount is more than 10% by weight, dissolving in the system tends to be difficult.

To the hair treating agent of the present invention, optional components besides the above components can be added accordingly as long as the effects of the present invention are not lost. The added components are mixed with water as the solvent. Examples of the components are cationic surfactants, nonionic surfactants, anionic surfactants, ampholytic surfactants, oily components, lubricants, antiseptics, chelating agents and perfumes.

These optional components are not particularly limited. Suitable examples of the cationic surfactant are tetraalkylammonium salts such as stearyl trimethyl ammonium bromide, stearyl trimethyl ammonium chloride, behenyl trimethyl ammonium chloride and distearyl trimethyl ammonium chloride. Suitable examples of the nonionic surfactant are polyoxyethylene cetyl ether, polyoxyethylene oleyl ether and polyoxyethylene castor oil. Suitable examples of the anionic surfactant are sodium polyoxyethylene laurylethersulfate, polyoxyethylene cetyletherphosphate and sodium α-oleate. Suitable examples of the ampholytic surfactant are 2-alkyl-N-carboxymethyl-N-hydroxyethyl imidazolinium betaine and amidopropyl betaine laurate.

Suitable examples of the oily component are vegetable oils such as avocado oil and castor oil, hydrocarbons such as liquid paraffin, light liquid isoparaffin and squalane, silicones such as high polymerization methyl polysiloxane and aminoethyl aminopropyl methyl siloxane-dimethylsiloxane polymer, and higher alcohols such as cetanol and stearyl alcohol.

Suitable examples of the lubricant are glycerin, polyethylene glycol, propylene glycol, dipropylene glycol, 1,3-butylene glycol and sorbitol.

Suitable examples of the antiseptic are methyl paraoxybenzoate, propyl paraoxybenzoate, sodium benzoate and phenoxy ethanol.

Suitable examples of the chelating agent are hydroxyethane diphosphoric acid and diethylene-triamine-pentaacetic acid pentasodium.

The form of the hair treating agent of the present invention is not particularly limited and various forms can be employed such as cream, lotion, gel, foam and spray. From the viewpoint of being easy to use, cream and gel are preferable.

In the process for straightening treatment of curly hair, after the reducing agent for straightening curly hair is applied, a knot test is conducted at the ends, the middle and the roots and the hair treating agent of the present invention is applied in the same amount as the reducing agent for straightening curly hair to areas that are sufficiently softened and then rubbed in to be thoroughly mixed. The reducing agent and the hair treating agent are rinsed off and the hair is dried. Subsequently, an oxidizing agent is applied and after combing with a rough comb while applying tension, the oxidizing agent is rinsed off.

The reducing agent for straightening curly hair that is used before applying the hair treating agent of the present invention is not particularly limited, but preferably contains an alkali agent. Preferable examples of the alkali agent are monoethanol amine, ammonium hydrogen carbonate and ammonia. The pH of the reducing agent for straightening curly hair is preferably 8 to 11. When the pH is less than 8, suppression of the reducing reaction by the hair treating agent is large and the hair may not be sufficiently softened. When the pH is more than 11, the hair and scalp tend to be damaged.

The oxidizing agent for straightening curly hair that is used after the applying the reducing agent for straightening curly hair and the hair treating agent of the present invention are washed off is not particularly limited. Hydrogen peroxide solution, sodium bromate and sodium perborate are preferably compounded as the oxidant and from the viewpoint of strong oxidization force, hydrogen peroxide solution is preferably compounded. To the oxidizing agent for straightening curly hair, for example, a pH buffer, a thickener and a surfactant can be added.

Hereinafter, the present invention is explained in detail by means of Examples, but the present invention is not limited thereto. In the following Examples, the amounts of the components are represented by “% by weight”.

### EXAMPLES 1 TO 7 AND COMPARATIVE EXAMPLES 1 TO 5

The hair treating agents of Examples 1 to 7 and Comparative Examples 1 to 5 were prepared according to the composition shown in Tables 1 and 2. Also, the reducing agent for straightening curly hair was prepared according to the composition shown in Table 3.

The hair treating agents were prepared by mixing each of the compounds according to the usual method. The reducing agent for straightening curly hair was also prepared by mixing each of the compounds according to the usual method.

TABLE 1

| | Amount (%) | | | | |
|---|---|---|---|---|---|
| | Com. | Com. | Com. | Com. | Com. |
| Components | Ex. 1 | Ex. 2 | Ex. 3 | Ex. 4 | Ex. 5 |
| Polyethylene | 2.0 | 2.0 | 2.0 | 2.0 | 2.0 |
| cetyl ether | | | | | |
| phosphate (20 E.O.) | | | | | |
| Liquid paraffin | 2.0 | 2.0 | 2.0 | 2.0 | 2.0 |
| Cetanol | 0.5 | 0.5 | 0.5 | 0.5 | 0.5 |
| Malic acid | — | — | 10.0 | — | 10.0 |
| Phosphoric acid | — | — | — | — | — |
| Sodium maleate | — | — | — | — | — |
| Sodium lactate | — | 7.0 | — | — | 7.0 |
| Copper gluconate | — | — | — | 1.0 | — |
| 1,3-butylene glycol | 1.0 | 1.0 | 1.0 | 1.0 | 1.0 |
| Purified water | 94.5 | 87.5 | 84.5 | 93.5 | 77.5 |

TABLE 2

| | Amount (%) | | | | | | |
|---|---|---|---|---|---|---|---|
| Components | Ex. 1 | Ex. 2 | Ex. 3 | Ex. 4 | Ex. 5 | Ex. 6 | Ex. 7 |
| Polyethylene | 2.0 | 2.0 | 2.0 | 2.0 | 2.0 | 2.0 | 2.0 |
| cetyl ether | | | | | | | |
| phosphate (20 E.O.) | | | | | | | |
| Liquid paraffin | 2.0 | 2.0 | 2.0 | 2.0 | 2.0 | 2.0 | 2.0 |
| Cetanol | 0.5 | 0.5 | 0.5 | 0.5 | 0.5 | 0.5 | 0.5 |
| Malic acid | 10.0 | 10.0 | 10.0 | — | 5.0 | 10.0 | 20.0 |
| Phosphoric acid | — | 7.0 | 7.0 | 10.0 | — | 5.0 | — |
| Sodium maleate | — | — | 7.0 | — | — | 3.0 | — |
| Sodium lactate | 7.0 | 7.0 | — | 7.0 | 3.0 | 3.0 | 15.0 |
| Copper gluconate | 1.0 | 1.0 | 1.0 | 1.0 | 1.0 | 1.0 | 1.0 |
| 1,3-butylene glycol | 1.0 | 1.0 | 1.0 | 1.0 | 1.0 | 1.0 | 1.0 |
| Purified water | 76.5 | 69.5 | 69.5 | 76.5 | 85.5 | 72.5 | 58.5 |

TABLE 3

| Components | Amount (%) |
|---|---|
| Ammonium thioglycolate | 8.5 |
| Monoethanol amine | 3.0 |
| Ammonium hydrogen carbonate | 1.0 |
| Ammonia water (25%) | 1.0 |
| Stearyl alcohol | 1.0 |
| Polyoxyethylene oleyl ether | 1.5 |
| Purified water | 84 |
| pH | 9.0 |
| Color of hair treating agent after preparation | white |

(Color of mixture of reducing agent and hair treating agent immediately after mixing and measurement of pH)

30 g of the reducing agent for straightening curly hair shown in Table 3 was mixed with 30 g of each hair treating agent of Comparative Examples 1 to 5 and Examples 1 to 7. The color of the mixture immediately thereafter and the pH were measured.

TABLE 4

| | Com. Ex. | | | | |
|---|---|---|---|---|---|
| | 1 | 2 | 3 | 4 | 5 |
| pH of hair treating | 7.0 | 7.2 | 1.7 | 4.3 | 3.3 |
| agent after preparation | | | | | |
| Color of hair treating | white | white | white | blue | white |
| agent after preparation | | | | | |
| pH of mixture after | 8.9 | 8.9 | 4.3 | 8.9 | 4.4 |
| mixing with reducing agent | | | | | |
| Color of hair treating | white | white | white | yellow | white |
| agent after mixing with | | | | | |
| reducing agent | | | | | |

TABLE 5

| | Ex. | | | | | | |
|---|---|---|---|---|---|---|---|
| | 1 | 2 | 3 | 4 | 5 | 6 | 7 |
| pH of hair treating | 3.3 | 2.2 | 2.2 | 2.0 | 3.2 | 2.4 | 3.5 |
| agent after preparation | | | | | | | |
| Color of hair treating | blue | blue | blue | blue | blue | blue | blue |
| agent after preparation | | | | | | | |
| pH of mixture after | 4.4 | 3.8 | 3.8 | 4.3 | 4.6 | 3.9 | 4.0 |
| mixing with reducing agent | | | | | | | |
| Color of hair treating | yellow | yellow | yellow | yellow | yellow | yellow | yellow |
| agent after mixing with | | | | | | | |
| reducing agent | | | | | | | |

As shown in Table 4, in Comparative Examples 1 to 3 and 5, it could not be determined whether the reducing reaction had ended and ending of the reducing reaction could not be visually observed. Also, in Comparative Example 4, the color changed even though the reducing reaction had not ended. In contrast, in Examples, the reducing reaction was found to have ended and ending of the reducing reaction could be visually observed.

According to the hair treating agent of the present invention, straightening treatment of curly hair can be uniformly conducted without damaging the hair, as the hair treating agent contains a pH buffer and an oxyacid metal salt and as a result, can end reducing reaction of the reducing agent for straightening curly hair and enables visual observation of the ending of the reaction.

What is claimed is:

1. A hair treating agent that is applied after applying a reducing agent for straightening curly hair to end the reducing reaction and enables visual observation of the ending of said reaction, comprising a pH buffer and an oxyacid metal salt.

2. The hair treating agent of claim 1, wherein pH is 2 to 4.

3. The hair treating agent of claim 1, wherein pKa of the acid composing said pH buffer is 2 to 4.

4. The hair treating agent of claim 1, wherein said oxyacid metal salt is copper gluconate and the amount thereof is 0.1 to 10% by weight.
